(19) United States
(12) Patent Application Publication
(10) Pub. No.: US 2013/0253366 A1
(43) Pub. Date: Sep. 26, 2013

(54) CORE BODY TEMPERATURE MEASURING DEVICE

(71) Applicant: Kenneth P. Gross, Lincoln, NE (US)

(72) Inventor: Kenneth P. Gross, Lincoln, NE (US)

(21) Appl. No.: 13/430,612

(22) Filed: Mar. 26, 2012

(51) Int. Cl. A61B 5/01

(52) U.S. Cl. 600/549

(57) ABSTRACT

The present invention relates to a device and method for measuring core body temperature. The device utilizes a wire-bound thermistor passing through an adjustable memory foam such that the thermistor is exposed. The adjustable feature of the memory foam permits it to slide back and forth along the wire. The thermistor, having a resistance which varies with temperature, is inserted into the aural canal of the ear of a person, or the nostril of a person, in such a way that the thermistor does not come into contact with the wall of the aural canal or ear, or the wall of the nostril, thereby ensuring comfort to the person whose core body temperature is to be measured. The memory foam permits the device to seal out ambient air during the measurement thereby ensuring an accurate temperature measurement.

### FIELD OF THE INVENTION

The present invention relates to a core body temperature measuring device and method.

### BACKGROUND OF THE INVENTION

Complete and proper body function is reliant on a core temperature between 36.5 to 38.5 degrees Celsius. A temperature above 41.5 degrees Celsius or below 33.5 degrees Celsius results in a rapid decline in proper body functioning. Unintentional hypothermia can result in many adverse effects for a person, including arrhythmias, coagulopathies, impaired renal function, impaired immune function resulting in increased wound infections, delayed anesthetic recovery, increased incidence of blood transfusions, decreased intracranial pressures, prolonged hospitalization, increased metabolic and oxygen demand due to shivering, and even death.

Core temperature can be a vague and confusing term, but a theoretical concept of this term does exist. This concept involves a central core where body temperature varies little as peripheral tissues surround and buffer it from the environment through temperature gradients. Due to the composition of highly perfused tissues with a uniform temperature, a core measurement provides the most accurate reflection of true body temperature. Core temperature is most commonly measured in the pulmonary artery, esophagus, nasopharynx and via contact with the tympanic membrane. The four methods of core temperature measurement mentioned commonly use thermistors to detect changes in temperature. These thermistor sensors have been found to be stable, accurate, and have a short response time to temperature changes.

The pulmonary artery catheter is considered the gold standard, providing continuous and the most accurate measurement of core body temperature as the catheter is surrounded by blood directly from the central circulation. However, these catheters are highly invasive, making them unnecessary and unavailable in most patients, unless the patient requires invasive hemodynamic monitoring.

Esophageal and nasopharyngeal probes also provide continuous monitoring, accuracy and precision via a thermistor due to their location near the left ventricle and the hypothalamus in the brain, respectively. However, as with pulmonary artery catheters, these probes are invasive and not tolerated well by awake patients. As a result they are not a viable option for core temperature measurement other than during general anesthesia.

While undergoing general anesthesia invasive properties of nasopharyngeal or esophageal probes are not concerning, making them acceptable devices for core temperature measurement. However, after emergence body temperature must be monitored by another method, with current practice usually involving infrared thermometry at the tympanic membrane or temporal artery. These methods are easily accessible, but do not provide continuous data and, as mentioned above with tympanic membrane measurements, temporal artery measurements can provide unreliable reflections of core body temperature.

The fourth common site of core temperature measurement is the tympanic membrane. This is accomplished by inserting the thermistor into the aural canal until the patient feels the tip touch the tympanic membrane. It is then most often secured to the external auditory meatus with cotton and tape. Tympanic membrane thermometry is considered an acceptable form of core temperature measurement as it receives its blood supply from branches of the internal carotid artery that supply blood to the thermoregulatory center in the hypothalamus as well as several branches of the external carotid artery. Therefore it is probably the best method as an indirect measure of brain temperature. There is little air movement possible in the ear canal due to the structure of the tube, so the tympanic membrane should be at the temperature of the arteries that supply the area. One of the most appreciated features of the tympanic membrane method is the cleanliness achieved with a disposable thermometer. Despite the numerous advantages to tympanic membrane thermometry, there are drawbacks. A major disadvantage to this technique is that it can be uncomfortable for the patient due to scratching, bleeding or perforation of the tympanic membrane. Slight pressure on the thermistor to maintain contact with the tympanic membrane may cause pain, making this unsuitable for most patients.

An ideal thermometer should have several characteristic traits. First, the inherent accuracy should be within ±0.2 degrees Celsius between different measurements at the same site. Secondly, the thermometer cannot be sensitive to outside influences such as air temperature changes or irrelevant areas of the body like limbs and skin. The device must be stable in terms of accuracy and calibration and the size should be appropriate for the area of use. Ideally, this device should be quick to place and easy to use.

In view of the foregoing, a device and method for measuring core body temperature is needed that provides continuous measurement, is accurate, comfortable, inexpensive, and sanitary, and which can be adapted for use at different sites of measurement.

### SUMMARY OF THE INVENTION

In one aspect of the invention, a core body temperature measuring device is provided. The device comprises a memory foam, a wire passing through the memory foam, said wire having first and second ends, a thermistor having a resistance which varies with temperature, and a temperature display means, wherein the thermistor is operatively connected to the first end of the wire, wherein the temperature display means is operatively connected to the second end of the wire, wherein the thermistor extends completely beyond the memory foam and is exposed, and wherein the memory foam is capable of being moved back and forth along the length of the wire.

In another aspect of the invention, a method for measuring core body temperature is provided. The method comprises providing a core body temperature sensing device, the device comprising a memory foam, a wire passing through the memory foam, the wire having first and second ends, a thermistor having a resistance which varies with temperature, and a temperature display means, wherein the thermistor is operatively connected to the first end of the wire, wherein the temperature display means is operatively connected to the second end of the wire, wherein the thermistor extends completely beyond the memory foam and is exposed, and wherein the memory foam is capable of being moved back and forth along the length of the wire, inserting the thermistor end of the wire of the device into the aural canal of the ear of a person whose core body temperature is to be measured such that the thermistor does not come into physical contact with the aural canal or ear, and such that the memory foam plugs into the ear and seals out the ambient air, and reading the core body temperature displayed on the temperature display means.

In another aspect of the invention, a method for measuring core body temperature is provided. The method comprises providing a core body temperature sensing device, the device comprising a memory foam, a wire passing securely through the memory foam, the wire having first and second ends, a thermistor having a resistance which varies with temperature, and a temperature display means, wherein the thermistor is operatively connected to the first end of the wire, wherein the temperature display means is operatively connected to the second end of the wire, wherein the thermistor extends completely beyond the memory foam and is exposed, and wherein the memory foam is capable of being moved back and forth along the length of the wire, inserting the thermistor end of the wire of the device into the nostril of the nose of a person whose core body temperature is to be measured such that the thermistor does not come into physical contact with the nostril or nose, and such that the memory foam plugs into the nose and seals out the ambient air, and reading the core body temperature displayed on the temperature display means.

### BRIEF DESCRIPTION OF THE DRAWINGS

The invention may best be understood by reference to the following description, taken in conjunction with the accompanying drawings. The drawings described are only schematic and are non-limiting. In the drawings, the size of some of the elements may be exaggerated or distorted and not drawn on scale for illustrative purposes.

FIG. 1 illustrates a core body temperature measuring device in accordance with various embodiments of the present invention.

### DETAILED DESCRIPTION OF THE INVENTION

The present invention will be described with respect to particular embodiments and with reference to certain drawings but the invention is not limited thereto. Description of the various embodiments detailed below is for understanding the invention, and it will be understood that the invention is not limited to the particular embodiments described herein, but is capable of various modifications, rearrangements and substitutions, which will now become apparent to those skilled in the art without departing from the scope of the invention. Therefore, it is intended that the following claims cover all such modifications and changes that fall within the spirit and scope of the invention.

Where an indefinite or definite article is used when referring to a singular noun e.g. “a” or “an,” “the,” this includes a plural of that noun unless something else is specifically stated.

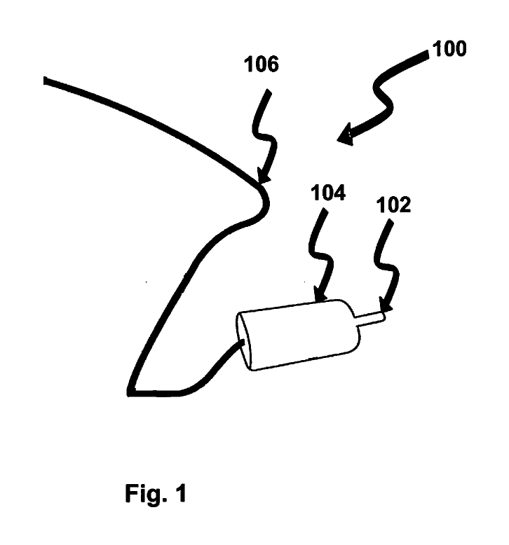

FIG. 1 is an illustration of a front view of the core body temperature measuring device of the invention. The device 100 comprises a memory foam 104, a wire 106 passing securely through the memory foam 104, said wire 106 having first and second ends, a thermistor 102 having a resistance which varies with temperature, and a temperature display means (not shown), wherein the thermistor 102 is operatively connected to the first end of the wire 106, wherein the temperature display means (not shown) is operatively connected to the second end of the wire 106, wherein the thermistor 102 extends completely beyond the memory foam 104 and is exposed, and wherein the memory foam 104 is capable of being moved back and forth along the length of the wire 106.

The memory foam 104 of the device 100 may be made of a material selected from the group consisting of polyurethane, polyethylene, polypropylene, and polystyrene. The memory foam 104 is preferably a size that permits insertion into the ear or nostril of a human subject while simultaneously sealing out ambient air. The memory foam 104 is adjustable with respect to the wire 106, and can be disposed of and replaced when required.

The wire 106 of the device 100 is preferably AWG gauge 20, 24, 30, or 36, and most preferably, AWG gauge 36. The wire 106 is preferably insulated in a dielectric insulator such as polyethylene, silicon rubber, polyvinylchloride, and tetrafluoroethylene.

The thermistor 102 of the device 100 is preferably coated by a phenolic or epoxy material, or encased in a tube composed of polyimide, polyvinylchloride, or glass.

The device and method of the present invention provide continuous and accurate monitoring of core body temperature, and further provide an inexpensive, disposable, noninvasive, and sanitary way to measure core body temperature comfortably for both awake subjects and subjects under general anesthesia.

Although the invention has been described with respect to one or more embodiments, equivalent alterations and modifications will occur to others skilled in the art upon the reading and understanding of this specification. In addition, while a particular feature of the invention may have been disclosed with respect to only one of several embodiments, such feature may be combined with one or more other features of the other implementations as may be desired and advantageous for any given or particular application.

### WORKING EXAMPLES

Temperature monitoring has an important place in the profession of anesthesia as numerous consequences exist as a result of the deviation from normothermia due to the anesthetic itself and environmental factors. Alterations in body temperature can have numerous detrimental effects on physiologic processes leading to abnormal function of tissue and organ systems.

Multiple monitoring sites and devices are used to measure temperature, each with benefits and limitations. Temperature monitoring sites can provide convenience and comfort for the patient and anesthesia professional at the expense of measuring core temperature. Other invasive sites are available to more accurately measure core temperature, however are inconvenient and can be uncomfortable for the patient.

A method that can prove to be a reliable measure of core body temperature as well as be tolerable in patients throughout all levels of anesthetic depth with current devices has proven difficult. The importance of finding this method is clear as evidenced by the outcomes resulting from the lack of maintenance of normothermia.

Current methods of temperature monitoring either accurately reflect core temperature but require invasive placement, such as the thermistor on pulmonary artery catheters, or are convenient for the anesthesia professional to access and comfortable for the patient, but are poor reflections of core body temperature, such as thermistors placed on the skin near the axillary artery. This presents a dilemma for the anesthesia professional as to whether the data collected is convenient for themselves and comfortable for the patient, or an accurate representation of core body temperature, yet more invasive.

The purpose of this study was to test an aural canal temperature probe that could be used to measure core body temperature as well as be comfortable for the patient throughout the operative course despite anesthetic technique employed. By comparing the aural canal temperature probe to another device which is a known accurate method of core body temperature measurement, this alternate probe may be confirmed as an appropriate measurement device.

The hypothesis tested was: Under general anesthesia, an aural canal temperature probe utilizing a thermistor embedded in a soft foam ear plug will measure within ±0.5 degrees Celsius (° C.) when compared to the same thermistor placed invasively in the nasopharynx. The approximate time that the difference between nasopharyngeal and aural canal temperature probe measurements stabilize will be observed as well.

The device to be tested in this study involves the same thermistor currently used for skin and nasopharyngeal temperature measurement passed thru a dense foam ear plug so that only the tip of the thermistor is protruding through the foam plug. The foam plug ensures isolation from outside influences and allows for stabilization of the thermistor preventing it from migrating toward the tympanic membrane or dislodging from the aural canal. The average aural canal depth in an adult ranges from 2.5 to 3 cm and this probe measures 2.4 cm in overall length with a variable portion remaining outside the aural canal, providing a theoretical built in safety cushion. Comparison of this device with nasopharyngeal temperature, which has been shown to be an accurate reflection of core temperature measurement, could establish it as an acceptable method of determining continuous, core body temperature in both anesthetized and awake patients.

To establish an aural canal temperature probe as an accurate measure of core temperature, it must be compared to an accepted site and method of core temperature measurement. This study was of a prospective descriptive comparison design in which each participant had data collected for the standard temperature measurement device, nasopharyngeal, as well as the experimental temperature measurement device, aural canal. This design allowed each participant to serve as their own control.

A convenience sample of 28 participants, as indicated by power analysis utilizing parameters of p<0.05, power=0.8, and an effect size of 0.34 at a community medical center was used. Patients scheduled for surgery that fit the inclusion criteria were approached by one of the two primary researchers for enrollment in the study.

Inclusion criteria for this study were: 19 years of age or older; minimum duration of general anesthesia of 30 minutes; airway securement via endotracheal tube (ETT) or laryngeal mask airway (LMA); ability to speak and understand English; and a willingness to consent to the research study. Exclusion criteria for this study were sinus surgery; oropharyngeal surgery; facial surgery; ear surgery; presence of skull fracture; ruptured tympanic membrane; nasotracheal intubation; pregnancy; and patient refusal.

Limitations of this study included relatively small sample size when compared with larger studies. All data was collected at one institution which could be viewed as a limitation. The subject pool may not have been representative of the general population as it was a convenience sample. The researchers were not blinded to the data as it was recorded. Short anesthetic durations may not have allowed the two probes to equilibrate with body temperature completely before being discontinued.

Initial assumptions included proper functioning of monitoring equipment and accurate recording of reported data. Although the sample size and demographics may not have been entirely representative of the general population, the researchers assumed that comparing the two sites of temperature measurement in each patient would provide adequate control and eliminate any differences due to demographic variables. It was assumed that the type of airway device employed, LMA versus ETT, the use of warming blankets, ambient room temperature, surgical procedure, anesthetic technique and anesthetic drug selection, and patient positioning would not affect the correlation of temperature between the two sites.

Prior to beginning the study, in order to support this presumed temperature variability between probes utilizing the same monitor, temperature data was gathered for a time period of thirty minutes with both probes exposed to ambient temperature. This process was repeated three times in total. During all three time periods, the maximum difference between the two probes was 0.1° C. at time periods of 0, 10, 21, and 30 minutes (Table 1). These values were well within the expected range of ±0.3° C. stated by the manufacturers.

Participants eligible for inclusion into the study were approached by one of the primary researchers for enrollment. The procedure for the study was fully explained to the participant, as well as risks, benefits and what participation would entail. Informed consent was then obtained. At this time, each participant was assigned a number used to reference the data collected during the anesthetic. The aural canal temperature probe was fitted in the participant's right ear to ensure that there was no discomfort to the participant. The probe was marked at the tragus of the ear so that it would be inserted to the same depth in the operating room. The probe was removed prior to transport to the operating room.

Participant preparation proceeded for the procedure. Induction of anesthesia and securement of the airway were completed by the anesthesia professional caring for the participant. Immediately after airway confirmation, the nasopharyngeal temperature probe was placed 3 to 4 cm into the right nare and the insulated wire secured to the cheek with the adhesive disk supplied by the manufacturer. Then the aural canal temperature probe was gently inserted into the right aural canal to the depth previously marked. Both of the temperature measurements were interpreted, displayed, and recorded by the same freestanding monitor. Temperature readings were recorded throughout general anesthesia at intervals of one minute for the first fifteen minutes and every three minutes thereafter. In addition to temperature measurements, data recorded included participant age, sex, and type of airway device used. Both temperature probes were removed prior to emergence from general anesthesia.

The same type of temperature probe was utilized for each monitoring site. This probe can be used to monitor skin, nasopharyngeal and esophageal temperature during surgery. All data was displayed and recorded throughout the case by a free standing monitor. The same free standing monitor, interchangeable module, and cable were used for all data collection with every patient to decrease variability due to published temperature performance specifications of the monitor.

In order to prevent movement in the aural canal the thermistor was immobilized by inserting it through a soft and compressible foam earplug prior to placement in the participant's ear. Only the tip of the thermistor emerged from the end of the ear plug, eliminating contact with structures in the aural canal. This foam plug helped prevent any advancement of the probe and provided a seal from environmental temperature influences.

Data was collected automatically by the freestanding monitor at one minute intervals throughout measurement times. This data was not saved by the monitor across subjects at the end of the surgical procedure. It was instead recorded onto a spreadsheet data tool created by the researchers. This spreadsheet included participant demographic information limited to age and sex. Type of airway device utilized was also recorded, and each participant was assigned an identification number. The temperature recording area included nasopharyngeal and aural canal temperatures at one minute intervals for the first fifteen minutes, and three minute intervals thereafter.

Analysis consisted of comparing the temperatures recorded by the nasopharyngeal probe to temperatures recorded by the aural canal probe. The temperatures compared were recorded at identical points in time by each probe. A t-test for paired data was used to evaluate the accuracy of temperature measurement in the aural canal compared to the nasopharyngeal temperature. The hypothesized outcome was to find no significant statistical difference in the two data sets, with significance set at p<0.05 and a confidence interval of less than ±0.5° C. The value of ±0.5° C. was chosen because it has been used in studies numerous times previously, and is an acceptable deviation between core temperature measurement sites. Another rationale is that this amount of temperature change has been sufficient to be associated with complications attributed to hypothermia. Precision was analyzed by calculating the correlation coefficient between the two measurements at the same time, with a hypothesized outcome that a strong relationship (R>0.61) exists between the values recorded at both sites.

The study population consisted of 28 individuals receiving general anesthesia for various procedures at a community medical center. Age of the participants ranged from 23 to 83 years old with the mean age of 61.2 years and a standard deviation (SD) of ±18.6 years (Table 2). Of the 28 participants, 42.9% (n=12) were male and 57.1% (n=16) were female (Table 3). Two airway devices were employed in this study, a standard endotracheal tube (ETT), and a disposable laryngeal mask airway (LMA). ETT's (n=24) were placed in 85.7% of the participants, while LMA's (n=4) were placed in 14.3% of the participants (Table 4).

During the data collection process, temperature readings for each probe were recorded at intervals of one minute for the first fifteen minutes, and every three minutes thereafter until the end of the surgical procedure. The minimum duration of general anesthesia for this study was 30 minutes to allow adequate time for the two different probes to equilibrate and ensure an adequate number of data points. Of the 28 participants, the minimum duration of anesthesia and thus data collection was 30 minutes, maximum was 93 minutes, with an average of 51.1 minutes. Data was collected on two additional participants enrolled in the study, however anesthetic duration was 21 minutes for each individual and this data was not included in analysis. Although temperature data was collected for the duration of general anesthesia in all participants, to maximize the statistical analysis for the data collected, only data collected during the initial 30 minutes were utilized. The 30 minute time frame was chosen, as all participants had temperature data points collected during this time period. For the purpose of statistical analysis, the temperature readings were analyzed at four benchmark time points where all participants had recorded data points. These points were times of 0, 10, 21, and 30 minutes. At these times a t test and Pearson correlation were utilized.

The minimum temperature measured by the nasopharyngeal temperature probe was 32.5° C. The maximum recorded nasopharyngeal temperature was 38.6° C. Minimum aural canal temperature measurement was 33.2° C. while the maximum was 38.2° C.

At the first benchmark time of 0 minutes, the minimum nasopharyngeal temperature recorded was 32.5° C. and the maximum was 38.6° C. The mean nasopharyngeal temperature was 36.3° C. with a SD of ±1.0 and a standard error of the mean (SEM) of ±0.19° C. Minimum aural canal temperature was 33.2° C. and the maximum was 37.0° C. The mean aural canal temperature was 35.2° C. with a SD of ±0.81 and a SEM of ±0.15° C. (Table 5). In 26 of the 28 participants the nasopharyngeal probe recording was the higher starting temperature.

At time 0 minutes, the minimum difference between nasopharyngeal and aural canal temperature measurement was 0.1° C. The maximum difference found between the two temperature probes was 3.3° C. The mean temperature difference between the two probes was 1.1° C. with a SD of ±0.95 and a SEM of ±0.18° C. A 95% confidence interval was calculated resulting in a lower value of 0.76 and an upper value of 1.5° C. A t ratio using the mean difference and SEM was calculated to be 6.3 resulting in a p value of 0.000 (Table 6). A Pearson correlation (r=0.47, p=0.013) between nasopharyngeal and aural canal temperatures was found (Table 7).

At the second benchmark time of 10 minutes, the minimum nasopharyngeal temperature recorded was 35.6° C. and the maximum was 38.5° C. The mean nasopharyngeal temperature was 36.4° C. with a SD of ±0.60 and a SEM of ±0.11° C. Minimum aural canal temperature was 34.7° C. and the maximum was 37.9° C. The mean aural canal temperature was 36.1° C. with a SD of ±0.60 and a SEM of ±0.11° C. (Table 5). In 23 of the 28 participants the nasopharyngeal probe recording was the higher temperature, and in four participants the temperature readings of both probes were equal.

At 10 minutes the minimum difference between nasopharyngeal and aural canal temperature measurements was 0.0° C., or equal measurements in both probes. Maximum difference between the two temperature probes was 1.1° C. The mean temperature difference between the two probes was 0.4° C. with a SD of ±0.29 and a SEM of ±0.05° C. A 95% confidence interval was calculated resulting in a lower value of 0.24 and an upper value of 0.46° C. A t ratio using the mean difference and the SEM was calculated to be 6.4 resulting in a p value of 0.000 (Table 6). A Pearson correlation (r=0.88, p=0.000) between nasopharyngeal and aural canal temperatures was found (Table 7).

Twenty-one minutes was the third benchmark time for data analysis. The minimum nasopharyngeal temperature recorded was 35.5° C. and the maximum was 38.6° C. The mean nasopharyngeal temperature was 36.3° C. with a SD of ±0.65 and a SEM of ±0.12° C. Minimum aural canal temperature was 34.9° C. and the maximum was 38.1° C. The mean aural canal temperature was 36.1° C. with a SD of ±0.58 and a SEM of ±0.11° C. (Table 5). In 20 of the 28 participants the nasopharyngeal probe recording was the higher temperature, and in three of the participants the temperature readings of both probes were equal.

At 21 minutes the minimum difference between the nasopharyngeal and aural canal temperature measurements was 0.0° C., or equal measurements in both probes. Maximum difference between the two temperature probes was 0.8° C. The mean temperature difference between the two probes was 0.3° C. with a SD of ±0.29 and a SEM of 0.06° C. A 95% confidence interval was calculated resulting in a lower value of 0.17 and an upper value of 0.40 ° C. A t ratio using the mean difference and the SEM was calculated to be 5.2 resulting in a p value of 0.000 (Table 6). A Pearson correlation (r=0.89, p=0.000) between nasopharyngeal and aural canal temperatures was found (Table 7).

At the final benchmark time of 30 minutes, the minimum nasopharyngeal temperature recorded was 35.6° C. and the maximum was 38.5° C. The mean nasopharyngeal temperature was 36.3° C. with a SD of ±0.64 and a SEM of ±0.12° C. Minimum aural canal temperature measurement was 35.0° C. and maximum was 38.1° C. The mean aural canal temperature measurement was 36.1° C. with a SD of ±0.61 and a SEM of ±0.11° C. (Table 5). In 21 of the 28 participants the nasopharyngeal probe recording was the higher temperature, and in three participants the temperature readings of both probes were equal.

At 30 minutes the minimum difference between temperature readings of the two probes was 0.0° C., or equal measurements in both probes. Maximum difference between the two temperature probes was 0.9° C. The mean temperature difference between the two probes was 0.3° C. with a SD of ±0.30 and a SEM of ±0.06° C. A 95% confidence interval was calculated resulting in a lower value of 0.16 and an upper value of 0.40° C. A t ratio using the mean difference and the SEM was calculated to be 4.9 resulting in a p value of 0.000 (Table 6). A Pearson correlation (r=0.88, p=0.000) between nasopharyngeal and aural canal temperatures was found (Table 7).

The 28 participants were a convenience sample selected based on inclusion and exclusion criteria outlined in the methods section above. The inclusion criteria requiring the participants to be 19 years of age or above, was chosen as 19 years is the legal age of consent. For informed consent purpose, the ability to speak and understand English was required by the researchers. Patients undergoing sinus, oropharyngeal, facial, and ear surgery were excluded due to proximity of probe placements to the surgical procedure. Presence of a skull fracture or known tympanic membrane rupture required exclusion due to the possibility of probe migration to unintended site. Participants undergoing nasotracheal intubation were excluded due to the requirement that the nasopharyngeal probe be placed in the right nare, limiting the anesthesia professional's selection of intubation site. Pregnant women were excluded from the study as they may be considered a vulnerable population.

As patients were approached to enroll in the study, no one refused to participate. One individual approached was deemed inappropriate for selection due to their inability to fully understand the purpose and procedure of the study. Two individuals who gave their consent to participate in the study did not have data collected due to time overlap with other participants already enrolled.

Participant data related to age and sex were gathered to gain an overall picture of the demographic characteristics of the study's population. The mean and SD for these two characteristics were reported in the findings, but were not used for specific statistical analyses of the temperature data recorded. Type of airway device utilized, ETT (n=24) and LMA (n=4) were recorded with the possibility of differences in level of airway seal impacting temperature recording at the nasopharyngeal site due to close physical proximity. However, the study population included many more participants with an ETT than an LMA making statistical analyses or generalizations about this potential phenomena difficult. Further investigation into this potential phenomena would require a larger study sample size with a more equal ratio of ETT to LMA use.

As displayed in the findings above, the mean difference between nasopharyngeal and aural canal temperature decreased over time from an initially greater mean difference of 1.1° C. at time 0 minutes, to lesser mean differences of 0.4, 0.3, and 0.3° C. at times 10, 21, and 30 minutes respectively. This result was not surprising as there was an expected time lag for the aural canal probe's foam plug to expand and seal the aural canal from outside influences allowing the air and foam plug to absorb body heat and provide a stable sampling environment. The wide mean difference (1.1° C.) at time zero along with a large SD (±0.95° C.) and lack of a strong correlation (r =0.47) shows that temperature measurements from the aural canal probe at time zero are not reliable for clinical use.

Standard deviations of the mean differences decreased sharply from an initial value of 0.95° C. at time zero to values of 0.29, 0.29, and 0.30° C. at times 10, 21, and 30 minutes respectively. This initial decrease and then stabilization of SDs for these times shows that at 10 minutes or more, the mean difference was more representative of the two probe's relationship than at time zero. When comparing these mean differences along with the SDs to the hypothesis that aural canal temperature would be within ±0.5° C. of nasopharyngeal temperature, it was found that a portion of our temperature data points fell outside of this expected range. At ten minutes, 68% of the aural canal data points were within the range of −0.1 to −0.7° C. and 95% of the aural canal data points were within the range of 0.2 to −1.0° C. when compared to nasopharyngeal data points. Similar values were found for times 21 and 30 minutes with the range for 68% of the aural canal data points being 0.0 to −0.6° C. and the range for 95% being 0.3 to −0.9° C. when compared to nasopharyngeal data points. Once again, these ranges of two SDs display some temperatures measured in the aural canal did not fall within the hypothesized ±0.5° C. when compared to nasopharyngeal temperatures. However when examining the raw data, 81% of the aural canal temperature measurements fell within the hypothesized ±0.5° C. from the time benchmark of 10 minutes and longer. At times 0, 10, 21, and 30 minutes t ratios (6.3, 6.4, 5.2, and 4.9) were greater than two and p values (0.013, 0.000, 0.000) were less than 0.05 indicating a significant statistical difference between nasopharyngeal and aural canal temperatures at these times. Though this statistically significant difference is present, when the raw data is examined and compared to other studies of similar design, the clinical difference may not be significant. In clinical practice it is considered that a temperature measurement method is reliable when the SD ranges from 0.3 to 0.5° C.

At time 0, the 95% confidence interval was much wider (0.76-1.5° C.) than subsequent benchmark times of 10, 21, and 30 minutes (0.24-0.46, 0.17-0.40, 0.16-0.40° C.) supporting the statement made previously in regards to time needed for aural canal sampling site equilibration. After this time point the 95% confidence interval narrowed and approached measured nasopharyngeal temperatures. From this data, it can be determined that repeated samples would have a 95% likelihood of having the mean temperature difference between nasopharyngeal and aural canal measurements fall within these intervals.

As described previously, the Pearson correlation (r=0.47) did not describe a strong relationship between nasopharyngeal and aural canal temperature at time 0 minutes. After this point, correlation was strong (r=0.88, 0.89, and 0.88) at times 10, 21, and 30 minutes respectively. This indicates from this time on, a strong linear relationship exists between the temperatures measured by the two probes. This correlation in itself does not indicate accurate measurement, nor does it imply that the aural canal temperature measurements fell within the hypothesized range of ±0.5° C. as compared to the nasopharyngeal temperatures. However it does indicate that temperatures measured within the aural canal with our device can be reliably associated with those measured in the nasopharynx, a known site for core temperature measure. The linear correlation seen in this study was expected as temperature values measured in any two sites are expected to correlate, but the strength of correlation was reassuring.

When looking at the participant pool, it was noted that none of the participants were markedly hypo or hyperthermic throughout the study. This is a potential gap in information as it is important to establish this probe's efficacy in all potential populations of use. It has been suggested in other studies that temperature extremes may be a limitation of tympanic thermometry. It may be possible to extrapolate that aural canal temperature measurement could be affected by temperature extremes as well. The manufacturer of the probe used in this study suggests that its reliability is acceptable from temperatures ranging from 5 to 45° C. Even with this manufacturer claim, it is still important to include this population in further testing.

Aural canal size variation across individuals is topic worthy of mention as well. It was noted during aural canal probe insertion that some participant's aural canals were tighter and slightly more difficult to place the probe in. This variance in fit across individuals may have led to spurious results in certain participants. In other studies, researchers have noted difficulty with probe placement due to narrow or odd shaped ear canals affecting repeatability. Specific participants during this study in which difficulty with probe insertion was encountered were not identified for investigation during analysis of statistics. It would be interesting to be able to look back at this group of participants and see how their aural canal measurements correlated to nasopharyngeal measurements in comparison to other participants.

Presence of large amounts of cerumen in the ear canal could potentially affect aural canal temperature measurement. This has been identified as a factor that can lower tympanic measurements and in previous studies had been criteria for exclusion from the study. Once again, tympanic measurements are not the same as the aural canal measurements taken in this study, but the possibility of cerumen affecting measurement values seems possible and even likely. One participant in this study was noted to have a large amount of cerumen stuck to the aural canal probe upon discontinuation of temperature measurement. Their mean difference between nasopharyngeal and aural canal temperature measurement for the time period of 10 to 30 minutes was 0.8° C. as compared to the overall mean difference across all participants of 0.3° C. during this time period. It seems possible that this presence of large amounts of cerumen in the auditory canal may have had a negative effect on accuracy of measurement and correlation in this participant.

Duration of measurement during general anesthesia during this study ranged from 30 minutes to 93 minutes. Thirty minutes was the cutoff for statistical analysis as this was the last time data points were available for all participants, and this time period seemed adequate to allow for probe equilibration. The equilibration and correlation between the two probes was steady after 10 minutes and up to 30 minutes in this study. It would be interesting to note the mean difference and correlation between the two probes throughout longer durations of anesthesia. Looking at the raw data available in this study beyond the 30 minute mark, these trends appeared to continue until measurement was discontinued. It is assumed that the strength of correlation between the two probes would maintain and possibly strengthen as measurement duration increased across nearly all individuals, but further study is needed to solidify this assumption on a broader basis.

Power analysis suggested that 28 participants was an adequate number for this study, however a larger sample size could identify outliers. The ability to isolate data for separate analysis that seems compromised due to poor fit, or large amounts of cerumen in the aural canal could prove beneficial. Identifying these factors' effect and extent of their effect could help with appropriate candidate selection in the clinical setting. The convenience sample did not allow for randomization as each participant served as his or her own control.

Currently there are no proven accurate, non-invasive methods for measuring core body temperature. Methods presently in use are either invasive requiring a general anesthetic or are comfortable and convenient but are not representative of core temperature. Commonly used acceptable sites for measuring core temperature during general anesthesia include the pulmonary artery, distal esophagus, and nasopharynx. By comparing the proposed aural canal temperature monitor to a known and accepted core temperature monitoring site, a method of core body temperature measure acceptable in all methods of anesthesia is possible.

Although statistical analysis revealed a significant difference between nasopharyngeal and aural canal temperatures at all four benchmark times, clinical significance of these differences must be taken into consideration. Mean differences between the two probes at times 10, 21, and 30 minutes were within the hypothesized ±0.5 degrees Celsius (° C.) and the majority (81%) of the aural canal data points fell within this range during the study. Though some of the aural canal data points fell outside of the two standard deviation (SD) range from the mean difference, the values found in this study were well within the hypothesized range, and ranges from previously cited studies of similar purpose and design.

The narrow confidence intervals at 10, 21, and 30 minutes support that repeated samples will have similar mean temperature differences between the two probes. Pearson correlations (r=0.88, 0.89, 0.88) at times 10, 21, and 30 minutes respectively demonstrated a strong linear relationship between the two probes. These further support the use of the aural canal as a core temperature measurement site, specifically with this probe. Further studies could add strength to this temperature monitoring method as a valid measure of core temperature.

One consideration for future studies includes an increase in sample size. Power analysis was used to guide sample size selection in this study, however an increase in sample size may enable the identification and isolation of any possible outliers. As described above there was a lack of any participants that were markedly hypothermic or hyperthermic in the sample. Although these changes are not anticipated to alter temperature correlation with the aural canal probe, further study including patients at these temperature ranges would support this assumption.

Temperature data points were gathered beyond 30 minutes for many of the participants but to analyze these values statistically, it would require the full sample population to have data recorded for these times. Thirty minutes appeared a sufficient time to allow equilibration to a steady correlative value between the two probes, and all raw data gathered beyond this point indicated a continued linear relationship. For future study it is recommended that a longer minimum anesthesia time is employed to further explore this.

The vast majority of this study's participants (n=24) had endotracheal tubes (ETT) placed for airway management. There was no intent to analyze any difference between temperature relationship across the ETT and laryngeal mask airway groups, however in future studies it would be suggested that these groups be closer to equal in numbers allowing for analysis of a possible relationship between airway type and temperature correlation between probes.

Use of forced air warming blankets is so prevalent in the operating room that any mode of temperature measurement should have its accuracy examined during concomitant use. If examined in future studies, attention must be paid to type of warming device, physical arrangement of the device on the body, temperature setting on the warming unit, and duration and intervals of warming. This may be of bigger concern with temperature devices that are not insulated from the influences of air currents from the warming device. Whether this is true or not, examination is warranted.

A commonly used temperature measurement method in monitored anesthesia care (MAC), regional, and neuraxial techniques is skin temperature measurement with a thermistor, often in the axilla. A study directly comparing axillary skin temperature with the two methods compared in this study could provide support for aural canal temperature measurement as a superior method to axillary skin temperature measurement. With this support, aural canal temperature measurement could be viewed as an acceptable and preferred method of temperature measurement in MAC, regional and neuraxial anesthesia. In addition to comparing the aural canal probe used in this study to an axillary skin probe, comparison to the commercially available aural canal device mentioned earlier would detect any differences in product function and accuracy.

In summary, this methods comparison study completed at a community medical center involving a 28 participant convenience sample revealed a possible alternative site and device for core temperature measurement. There are currently no devices available for non-invasive accurate measure of core temperature. This gap was identified and targeted for study. When clinical ranges are considered, data suggests a viable site and device for core temperature measurement even though statistical difference in the two sites of measurement exists. Further adjustment to the study design could strengthen the argument for this method as a standard measure of core temperature in all types of anesthetics.

TABLE 1
Ambient Temperature Trials

| | | |
|---|---|---|
| Trial | Probe | Temperature (° C.) |

| | | | | | |
|---|---|---|---|---|---|
| 1 | 1 | 17.2 | 17.7 | 17.4 | 17.7 |
| | 2 | 17.2 | 17.7 | 17.4 | 17.7 |
| 2 | 1 | 17.7 | 17.3 | 17.6 | 17.4 |
| | 2 | 17.6 | 17.2 | 17.5 | 17.3 |
| 3 | 1 | 17.7 | 17.3 | 17.2 | 17.3 |
| | 2 | 17.8 | 17.3 | 17.3 | 17.3 |

TABLE 2
Age Characteristics of Sample

| | Minimum | Maximum | Mean | SD |
|---|---|---|---|---|
| Age (years) | 23 | 83 | 61.2 | 18.6 |

TABLE 3
Sex Characteristics of Sample

| | Frequency | Percentage |
|---|---|---|
| Male | 12 | 42.9 |
| Female | 16 | 57.1 |
| Total | 28 | 100 |

TABLE 4
Airway Devices

| | Frequency | Percent |
|---|---|---|
| Endotracheal Tube | 24 | 85.7 |
| Laryngeal Mask Airway | 4 | 14.3 |
| Total | 28 | 100 |

TABLE 5
Paired Samples Statistics for Nasopharyngeal
and Aural Canal Temperature Probes

| | | | Temperature (° C.) | | | | |
|---|---|---|---|---|---|---|---|
| Time | Temperature | | Mini- | Maxi- | | | |
| (min) | probe | N | mum | mum | Mean | SD¹ | SEM² |
| 0 | Nasopharyngeal | 28 | 32.5 | 38.6 | 36.3 | 1.00 | 0.19 |
| 0 | Aural Canal | 28 | 33.2 | 37.0 | 35.2 | 0.81 | 0.15 |
| 10 | Nasopharyngeal | 28 | 35.6 | 38.5 | 36.4 | 0.60 | 0.11 |
| 10 | Aural Canal | 28 | 34.7 | 37.9 | 36.1 | 0.60 | 0.11 |
| 21 | Nasopharyngeal | 28 | 35.5 | 38.6 | 36.3 | 0.65 | 0.12 |
| 21 | Aural Canal | 28 | 34.9 | 38.1 | 36.1 | 0.58 | 0.11 |
| 30 | Nasopharyngeal | 28 | 35.6 | 38.5 | 36.3 | 0.64 | 0.12 |
| 30 | Aural Canal | 28 | 35.0 | 38.1 | 36.1 | 0.61 | 0.11 |

¹SD = standard deviation;
²SEM = standard error mean

TABLE 6
Paired Samples Test for Nasopharyngeal and Aural Canal Temperature Probes

| | | | Paired Differences | | | | | |
|---|---|---|---|---|---|---|---|---|
| | Minimum | Maximum | Mean | | | | | |
| | temperature | temperature | temperature | | | | | |
| Time | difference | difference | difference | SD | SEM | | | |
| (min) | (° C.) | (° C.) | (° C.) | (° C.) | (° C.) | 95% CI | t | p |
| 0 | 0.1 | 3.3 | 1.1 | 0.95 | 0.18 | [0.76, 1.50] | 6.3 | 0.000** |
| 10 | 0.0 | 1.1 | 0.4 | 0.29 | 0.05 | [0.24, 0.46] | 6.4 | 0.000** |
| 21 | 0.0 | 0.8 | 0.3 | 0.29 | 0.06 | [0.17, 0.40] | 5.2 | 0.000** |
| 30 | 0.0 | 0.9 | 0.3 | 0.30 | 0.06 | [0.16, 0.40] | 4.9 | 0.000** |

¹SD = standard deviation;
²SEM = standard error mean;
CI = confidence interval
**significant at p < 0.01 (2-tailed)

TABLE 7
Paired Samples Correlation

| Time (minutes) | N | R | p |
|---|---|---|---|
| 0 | 28 | .47 | 0.013* |
| 10 | 28 | .88 | 0.000** |
| 21 | 28 | .89 | 0.000** |
| 30 | 28 | .88 | 0.000** |

*correlation significant at p < 0.05 (2-tailed)
**correlation significant at p < 0.01 (2-tailed)

What is claimed is:

1. A core body temperature measuring device comprising:
a memory foam;
a wire passing through the memory foam, said wire having first and second ends;
a thermistor having a resistance which varies with temperature; and
a temperature display means,
wherein the thermistor is operatively connected to the first end of the wire, the temperature display means is operatively connected to the second end of the wire, the thermistor extends completely beyond the memory foam and is exposed to ambient air, and the memory foam is capable of being moved back and forth along the length of the wire.

2. A method for measuring the core body temperature of a person comprising:
providing a core body temperature sensing device, the device comprising a memory foam, a wire passing through the memory foam, the wire having first and second ends, a thermistor having a resistance which varies with temperature, and a temperature display means, wherein the thermistor is operatively connected to the first end of the wire, the temperature display means is operatively connected to the second end of the wire, the thermistor extends completely beyond the memory foam and is exposed to ambient air, and the memory foam is capable of being moved back and forth along the length of the wire;
inserting the thermistor end of the wire of the device into the aural canal of the ear of a person whose core body temperature is to be measured such that the thermistor does not come into physical contact with the aural canal or ear, and such that the memory foam plugs into the ear and seals out the ambient air; and
reading the core body temperature displayed on the temperature display means.

3. A method for measuring the core body temperature of a person comprising:
providing a core body temperature sensing device, the device comprising a memory foam, a wire passing securely through the memory foam, the wire having first and second ends, a thermistor having a resistance which varies with temperature, and a temperature display means, wherein the thermistor is operatively connected to the first end of the wire, the temperature display means is operatively connected to the second end of the wire, the thermistor extends completely beyond the memory foam and is exposed to ambient air, and the memory foam is capable of being moved back and forth along the length of the wire;
inserting the thermistor end of the wire of the device into the nostril of the nose of a person whose core body temperature is to be measured such that the thermistor does not come into physical contact with the nostril or nose, and such that the memory foam plugs into the nose and seals out the ambient air; and
reading the core body temperature displayed on the temperature display means.
